(19) United States
(12) Patent Application Publication
(10) Pub. No.: US 2006/0100582 A1
(43) Pub. Date: May 11, 2006

(54) Indwelling catheter arrangement

(76) Inventors: Martin Marianowicz, Munchen (DE); Ralf Klein, Velbert (DE); Jan Henke, Herten (DE); Herbert Feit, Wachtersbach (DE); Michael Haas, Bad Orb (DE); Reinhard Kruse, Gelnhausen (DE)

Correspondence Address: DENNISON, SCHULTZ, DOUGHERTY & MACDONALD, 1727 KING STREET, SUITE 105, ALEXANDRIA, VA, 22314, US

(21) Appl. No.: 10/489,614

(22) PCT Filed: Aug. 9, 2002
(86) PCT No.: PCT/EP02/08961

§ 371 Date: Apr. 23, 2004

(30) Foreign Application Priority Data
Sep. 24, 2001 (DE) 101 46853.9

(51) Int. Cl. A61M 5/178

(52) U.S. Cl. 604/158; 604/264

(57) ABSTRACT

An indwelling catheter arrangement includes a cannula, the proximal end of which starts from a receiver and the distal end of which has an opening, as well as a flexible catheter, which can be guided through the receiver and through the cannula opening. A guiding element extends during the placing of the catheter, the guiding element remaining in the catheter after the placement and being fixed in a defined position in relation to the catheter. This arrangement provides a catheter which has a desired flexibility and, simultaneously, cannot slip in an uncontrolled manner after placement.

The invention relates to a dwelling catheter arrangement comprising a cannula whose proximal end extends from a receptacle and whose distal end is equipped with an opening, in particular a Tuohy's cut with a side opening, as well as a flexible catheter that can be guided through the receptacle and through the opening, which in turn contains a guiding element during placement of the catheter.

Medications can be dispensed with a corresponding indwelling catheter arrangement into the epidural space of the patient over the desired time period. The catheter remains in the epidural space in that area where the medicinal treatment is supposed to take place. First the cannula is inserted over the hiatus sacralis or in the area of the vertebra tier into the epidural space for the positioning of the catheter and then the cannula is placed with the catheter. In order to guide it sufficiently and in order to permit the necessary stiffness for the shifting into the epidural space a guiding element such as a guiding wire that is removed after positioning of the catheter is contained in the catheter. This creates the risk that the catheter's position is changed when pulling it out.

During the positioning of the catheter it should have the desired flexibility in the distal area. For this, a dwelling catheter arrangement distributed by Epimed International, Inc. provides a catheter containing a flat spiral spring. The flat spiral spring is located at the distal end at a distance from the tip in order to achieve on the one hand the desired and to allow on the other hand liquid that is guided through the catheter to exit. After the catheter is positioned correctly the guiding wire, which during placement of the catheter runs therein, is removed.

The disadvantage of the appropriate flat spiral spring catheter is that the flat spiral spring can shear or get caught at the opening of the catheter comprising a cut section when the catheter is pulled out of the epidural space while the cannula still remains in place, leading to a stretching of the flat spiral spring. This, in turn leads to an extensive removal of the “stretched out flat spiral spring” from the body, which can lead health-related damages.

The present invention is based on the problem to further develop an indwelling catheter arrangement of the above-named type in such a way that the catheter has the desired flexibility in the distal area, while ensuring at the same time that no undesired repositioning of the positioned catheter takes place. The unproblematic removal of the catheter after completion, in particular of a medicinal treatment, should also be possible. If necessary the possibility should exist to affect stimulation in the area of the distal catheter end.

According to the invention the problem is essentially solved in leaving the guiding element in the catheter after having positioned said catheter and by fixing it in a relative position thereto. In doing so the guiding element, such as a wire or a pipe, should be fixated in a first position relative to the catheter during positioning of said catheter and fixated in a second position after the catheter has been properly positioned. Thus, the possibility exists that the distal end of the catheter has a different flexibility during the positioning of the catheter than during the administration of the medication. Independently from this, no undesired shifting of the catheter can take place as the guiding element remains in the catheter during the entire treatment.

It is, of course, also possible that the first and second positions are identical.

The guiding element, in particular, should in principal run within the catheter. In order to achieve an electrical stimulation, the guiding element can also extend through the distal end of the catheter and can protrude it.

The catheter preferably consists of a tube made of synthetic such as polyamide, polyurethane, silicone or similar materials allowing insertion through the cannula without any problems or its removal after the treatment has been completed. Here the catheter can also have a closed end. Openings, such as holes, can be provided for the medication to exit in the distal area. A corresponding number or a larger number of openings is provided if an electrical stimulation should take place via the guiding element, without a distal protrusion of the guiding element over the catheter.

In a further development of the invention it is provided that the indwelling catheter arrangement has in particular in some areas a pipe-shaped bracket, in which on one hand the guiding element is fixated and on the other hand the catheter runs with its proximal end, which is displaceable in its bracket. After adjusting the catheter along the guiding element, the catheter is preferably fastened with a clamping element. Using this method a relative fixation takes place between the catheter and the guiding element.

In particular, the catheter is fixed via a Tuohy burst adapter or another appropriate adapter that facilitates the required clamping, whereby the catheter can be fixated via a rubber element by means of squeezing.

The bracket is equipped with a liquid connection that is used for the supply of the dispensed medication so that it can flow to the desired location in the epidural space via the bracket and the catheter. The liquid connection offers the opportunity to add a standardized filter. The fluid outlet opening or openings at the distal area of the catheter can run inside of the catheter walls or through a catheter with an open end.

A design of the invention that should be especially highlighted has a bracket with a Y or V shaped geometry with a first leg running along a straight line and a second leg at an angle merging into it, whereby the first leg fixates the guiding element and the proximal end of the catheter is displaceable arranged therein. Here the guiding element can be fixated in the wall area on the face of the first leg, located at a distance in relation to the cannula.

According to another suggestion, the guiding element can be fixated in a proximal area of the bracket through clamps, in particular through clamps with a bent end of the guiding element.

As an alternative, the bracket could be a pipe element in which the guiding element is fixated. Here the guiding element should extend from a cylindrical bracket element positioned in the guiding element that in turn has at least one liquid passage. The liquid passage consists of several passages located in a cross-sectional view on a ring and concentrically surrounding the guiding element.

The guiding element preferably consists of a shape memory alloy such as a nickel-titanium alloy or should contain it.

If the guiding element is a wire, it should have a diameter between 0.30 mm and 0.55 mm whereby the catheter should have an outer diameter of 0.8 to 2.3 mm.

The guiding element could also be designed as a pipe.

Here the outer diameter must be adjusted to the lumen of the catheter in such a way that the guiding element is displaceable and that the fluid can reach the catheter tip through the lumen of the guiding element.

According to another design of the invention that should be especially stressed, a stimulation, such as a stimulation of the spinal cord, can take place using the indwelling catheter arrangement in that the guiding element consisting of an electrically conductive material, in particular consisting of memory material, is connected with a pole of a power source in order to affect the desired stimulation in the distal area of the catheter where the end of the guiding element is located after the catheter is positioned In other words, the stiffness of the catheter is not only adjusted on purpose but in addition, the guiding element also has the function of a mono-polar stimulation electrode.

In order to facilitate the electrical stimulation, the guiding element either extends through the catheter distally or is equipped with several openings in the distal area if it runs within the catheter in order to reach an electrically conductive contact or an electrically conductive connection to a tissue or to tissue fluid.

In particular, eight to twelve openings are provided with a closed catheter, in order to ensure the required scope of the necessary current conduction between the guiding element and the tissue.

It is especially provided for that the guiding element is fixated in a bracket preferably having a V or Y geometry, that the bracket has a first straight-lined leg along which the guiding element as well as the catheter that surrounds it and is displaceable thereto runs sectionally or in its direction, and that the bracket has a second leg that runs at an angle to the first leg where the guiding element is fixated. In doing so, the guiding element can extend through a steadfast fluid-tight plug in the bracket and can be connected outside of the bracket with an electrical contact. According to an alternative suggestion, the guiding element can be fixated in the bracket via an element, such as a plug, that closes the opening of the second leg of the bracket, wherein the plug in turn closes the opening in a fluid-tight manner, using for example glue.

In a further suggestion, the connection between the proximal area of the guiding element and the electrical conductor is injection-molded with synthetic, whereby it is formed into a plug that is liquid-tight and can be inserted into an opening of the bracket.

The guiding element can also extend from a metallic sleeve, which in turn can be inserted in an opening of the bracket in a liquid-tight manner.

Additional details, advantages and characteristics of the invention result not only from the claims and their characteristics—for themselves and/or in combination—but also from the following descriptions of the preferred embodiments shown in the drawings.

They show:

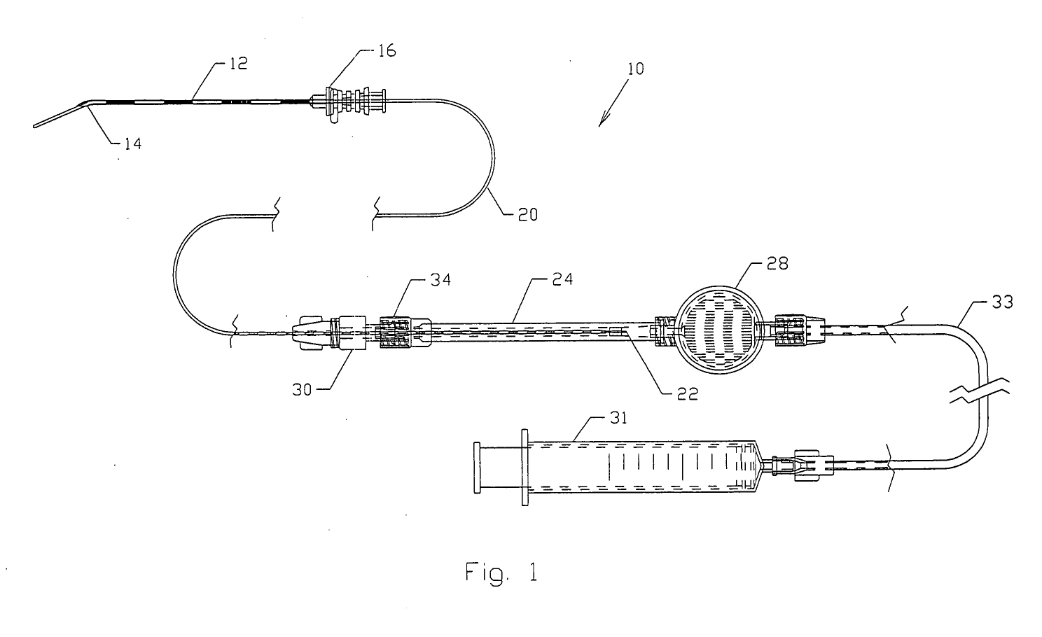

FIG. 1 a first embodiment of an indwelling catheter arrangement in a state that allows its usage,

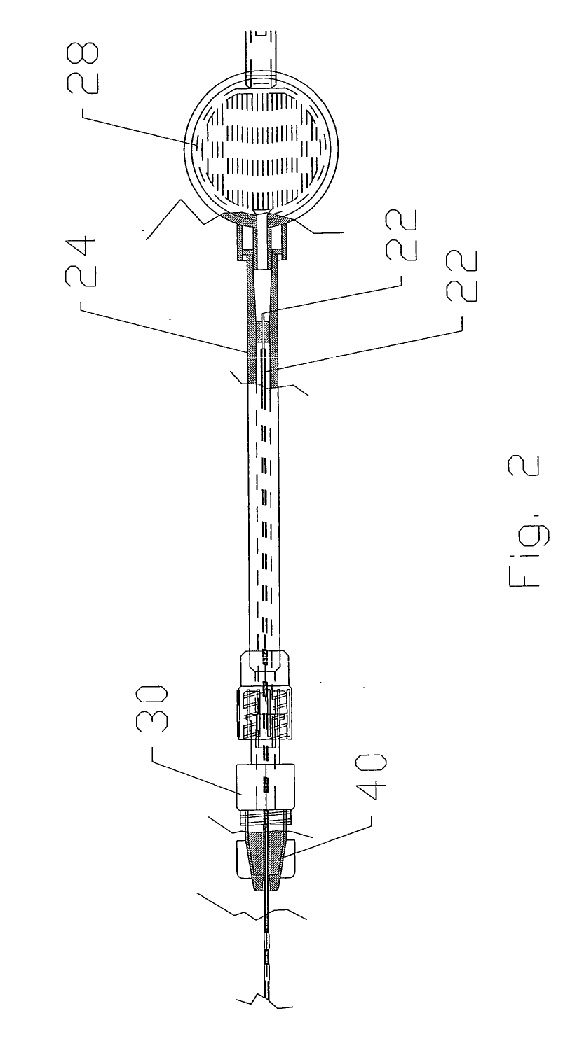

FIG. 2 a magnified representation of a bracket for a guiding element shown in FIG. 1,

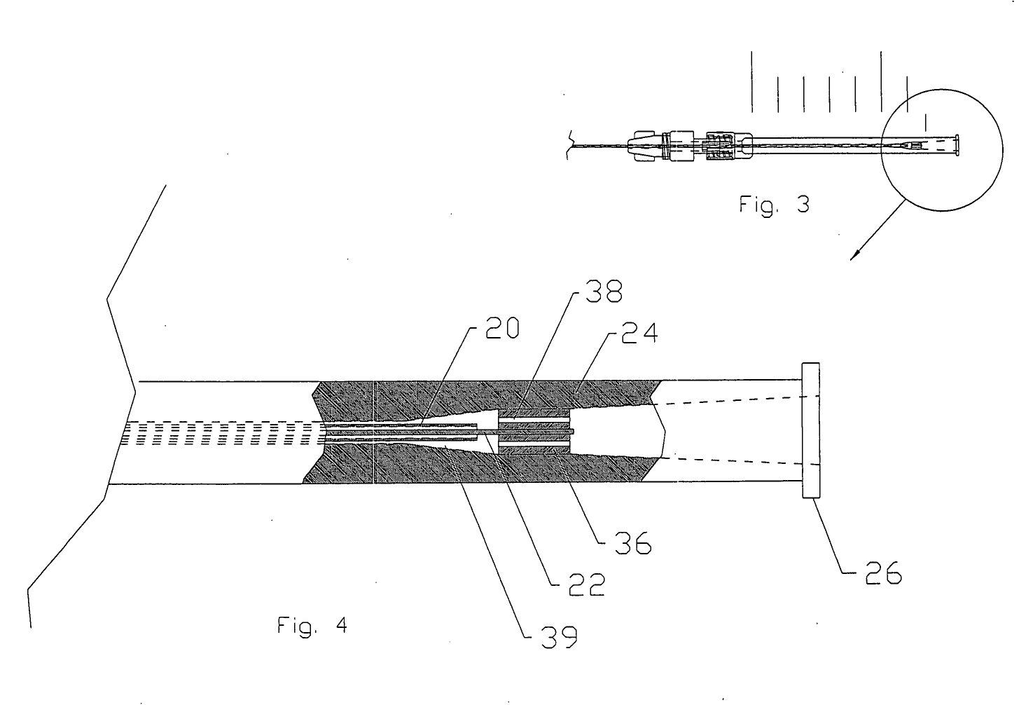

FIG. 3 a minimized representation of the bracket according to FIG. 2,

FIG. 4 a detail of the bracket according to FIGS. 2 and 3,

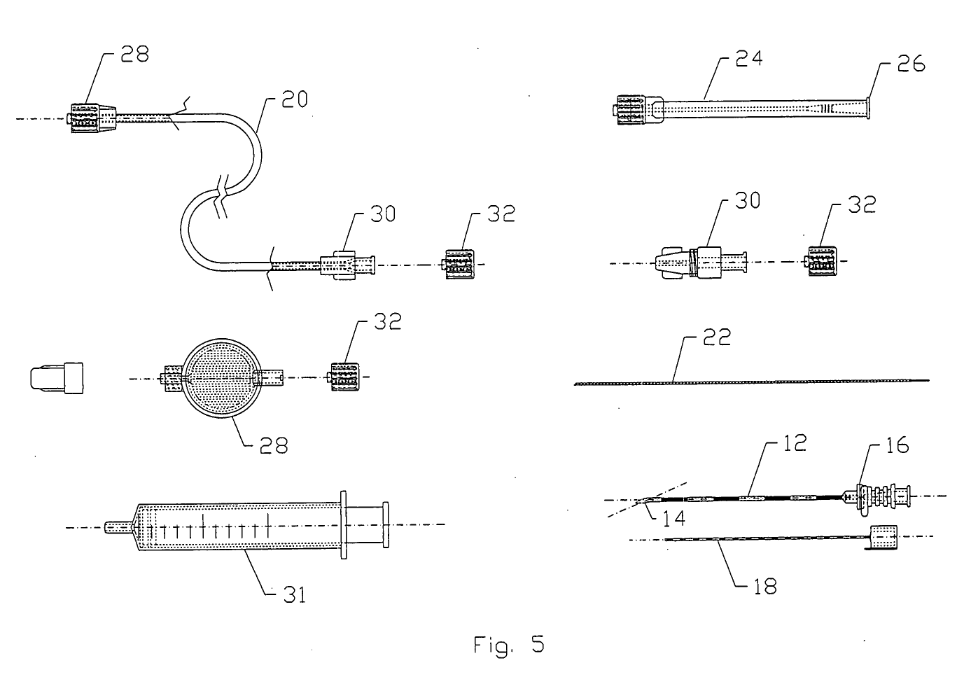

FIG. 5 individual parts of the indwelling catheter arrangement according to FIG. 1,

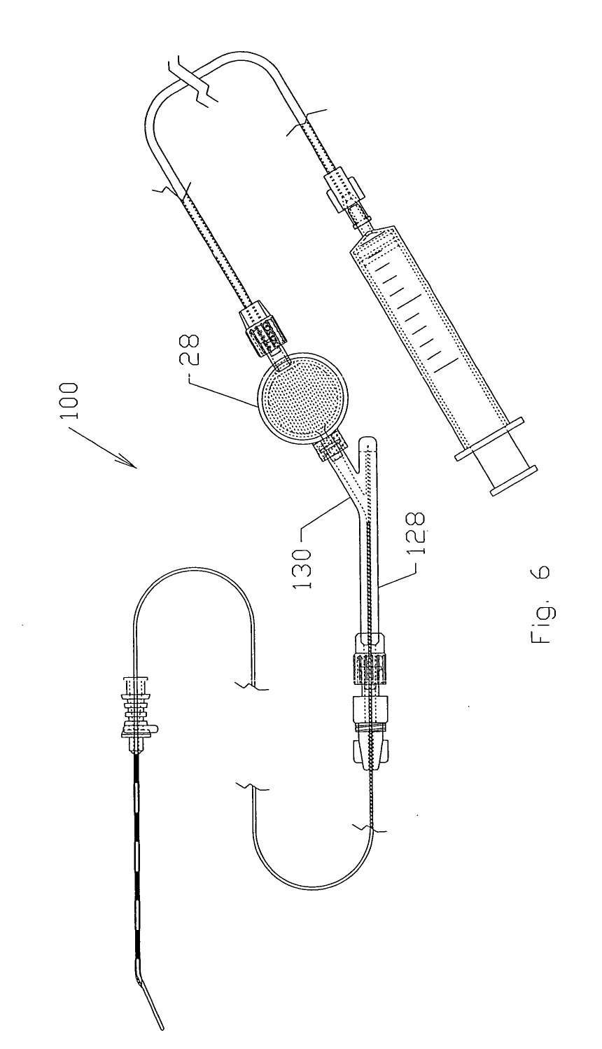

FIG. 6 a second embodiment of an indwelling catheter arrangement,

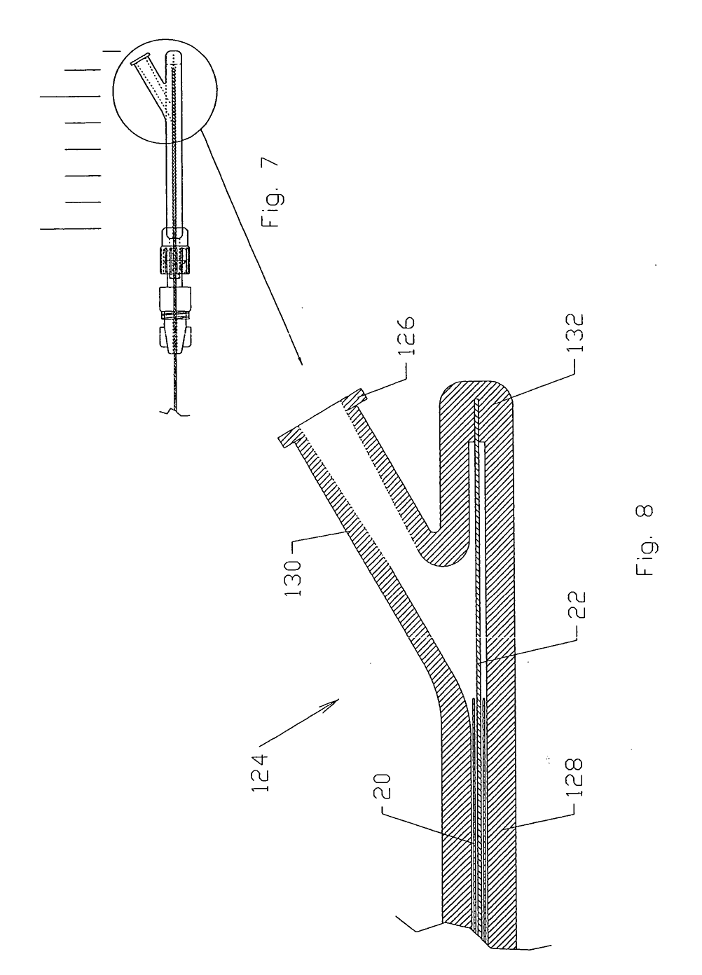

FIG. 7 a bracket for the guiding element shown in FIG. 6,

FIG. 8 a detail of the bracket according to FIG. 7,

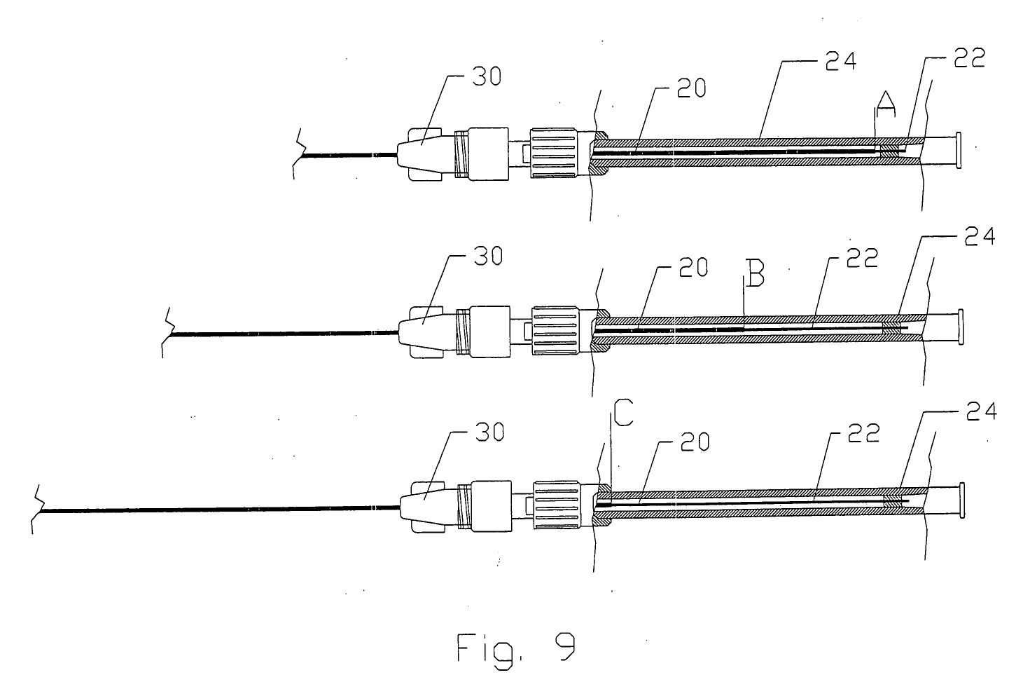

FIG. 9 various positions of a catheter in relation to a guiding element

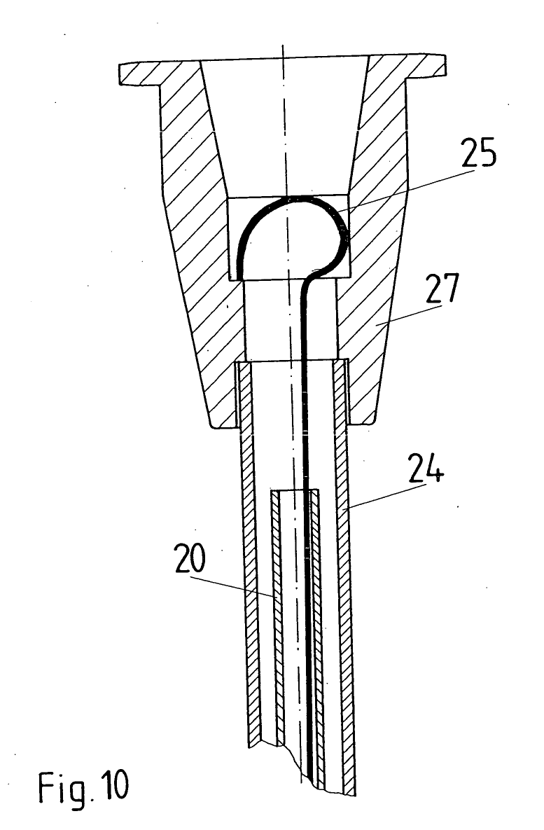

FIG. 10 an embodiment of a guiding element fixation,

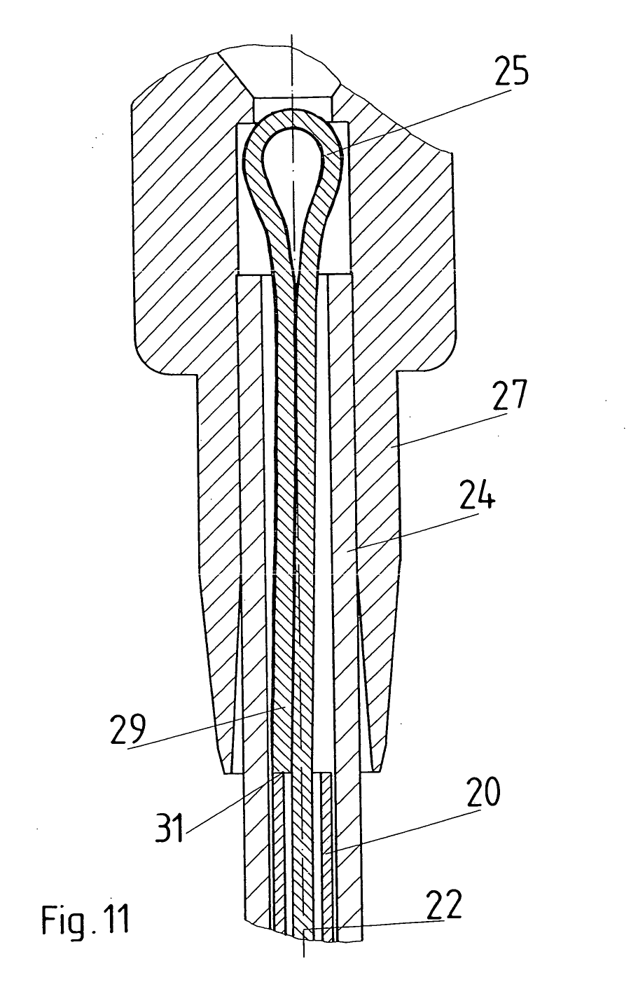

FIG. 11 an additional embodiment for the fixation of the guiding element,

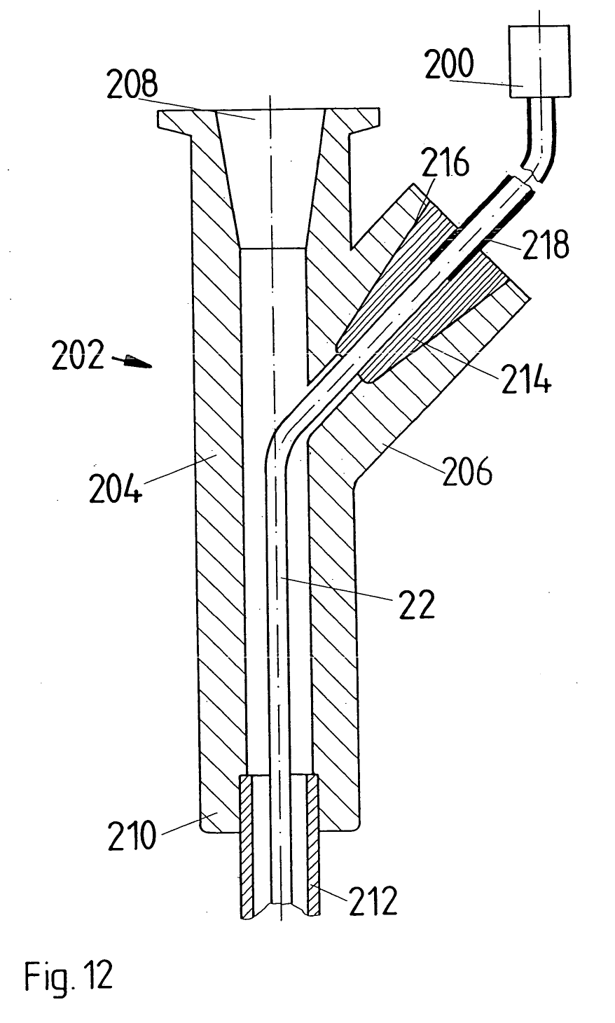

FIG. 12 a first embodiment of a guiding element fixated in a bracket with an electrical connection,

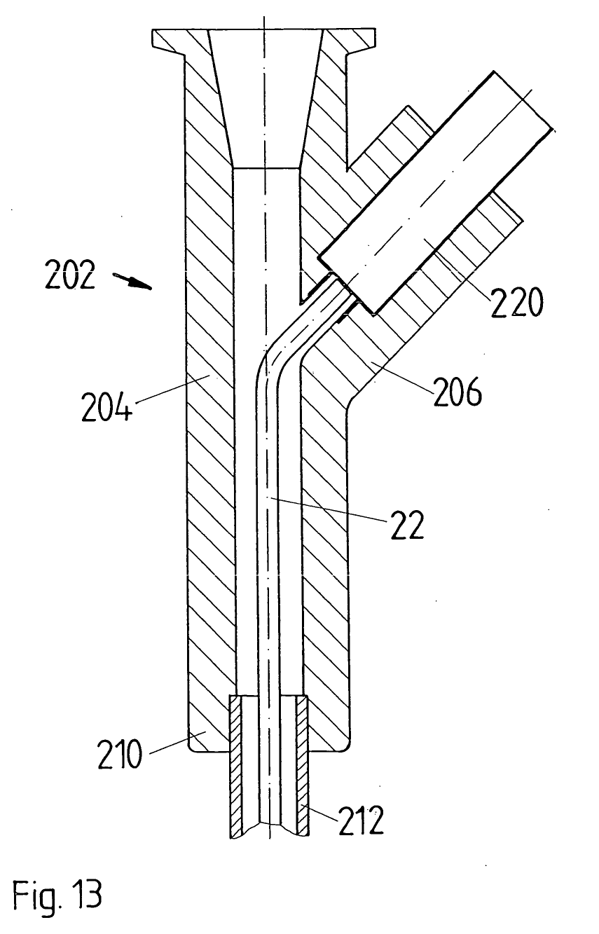

FIG. 13 a second embodiment of a guiding element fixated in a bracket with an electrical connection,

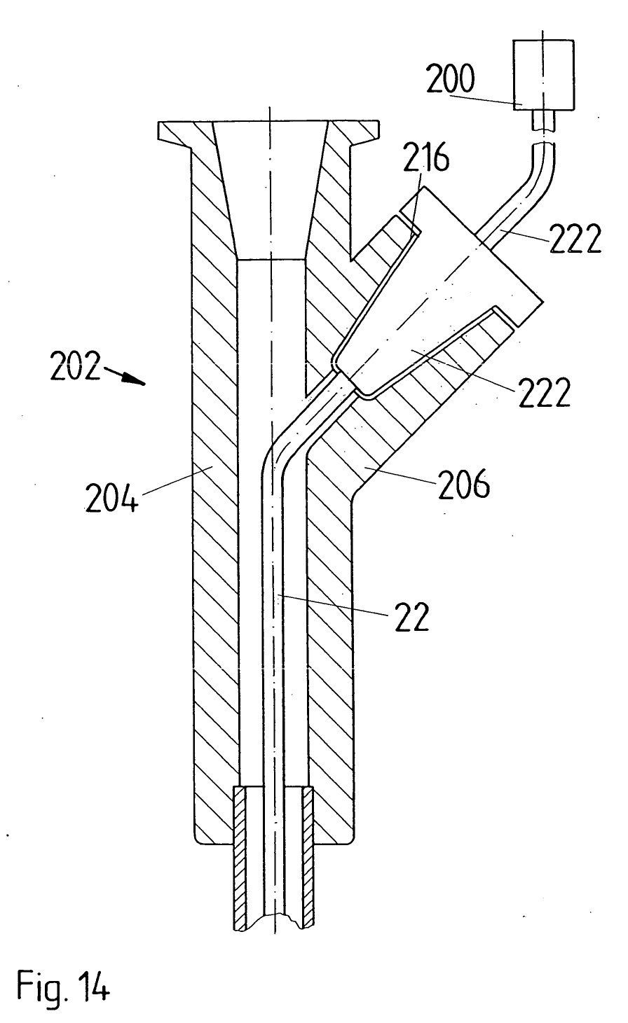

FIG. 14 a third embodiment of a guiding element fixated in a bracket with an electrical connection,

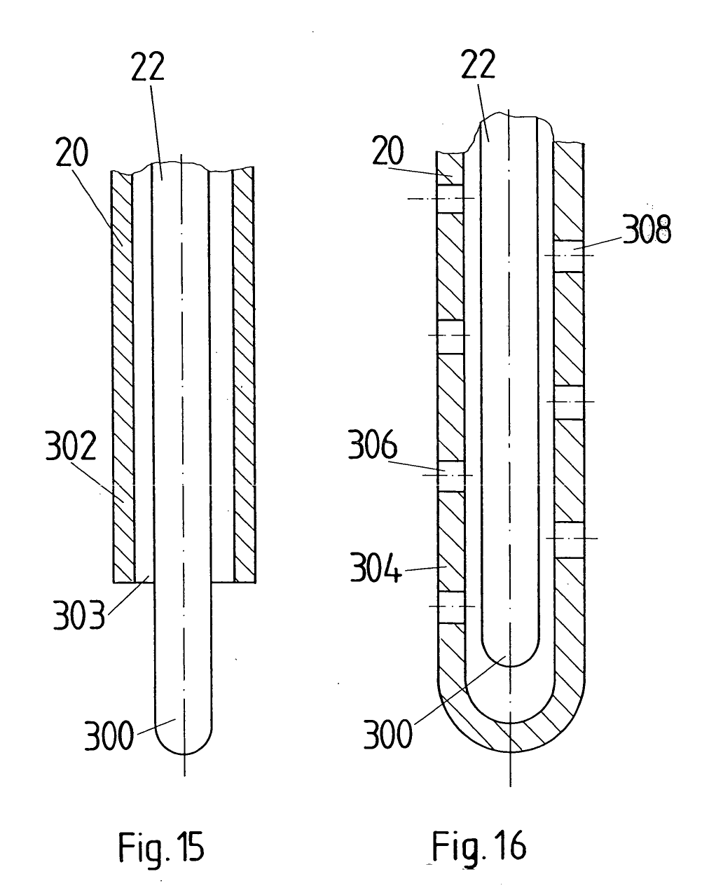

FIG. 15 distal area of a catheter in a magnified representation with the guiding element extending therethrough,

FIG. 16 a magnified representation of another embodiment in the distal area with a guiding element running inside.

Embodiments of indwelling catheter arrangements 10 or 100 can be seen in the Figures, where in principal the same elements have the same reference numbers, with which medications can be dispensed in the epidural space of a patient in the desired area. The indwelling catheter arrangements 10, 100 consist of known elements. For example, the indwelling catheter arrangement 10, 100 has a cannula 12, such as a split cannula, with preferably a Tuohy's cut and with an opening 14 on the side at the distal end. The proximal end extends from a receptacle with a central bore in order to close the cannula 12 with a mandarin 18, if so desired, or to connect the receptacle 18 with a catheter 20 in form of a plastic tube, made, for example, of polyamide. A guiding element 22 in form of a wire or a pipe runs within the tube 12 causing the catheter 20 to achieve the desired stiffness.

In order to facilitate a defined relative displacement between the guiding element 22 and the catheter 20, the guiding element 22 extends from a bracket 24 or 124, which in turn is equipped with a connection 26, 126 that can be connected with a filter element 28 through which the medication to be dispensed flows. The medication is dispensed via a commercial syringe 31 and a connecting tube 33, if necessary.

The catheter 20 can be introduced into the epidural space via the receptacle 16 of the cannula 12. The opposite end of the catheter 20 extends through a Tuohy burst adapter 20, which, in turn, can be connected with the bracket 24, 124.

Furthermore, the respective connections or openings can be sealed the usual way with sealing caps 32.

In order to achieve the desired stiffness at the distal end of the catheter 20, the guiding element 32 in form of a wire or a pipe can be fixated relative to the catheter 20 whereby its distal end can have a distance to the distal end of the catheter 20, especially when the catheter 20 has been placed. In order for the guiding element 22 to assume a defined position relative to the catheter 20, which provides—as mentioned earlier—the flexibility to the distal end of the catheter 20, the guiding element 22 extends from the bracket 24 and is fixated according to the embodiments of FIG. 1 to 5 in its distant cannula end via a cylindrical clamping element 36. In order to facilitate the flow of the fluid that is supplied via the filter element 28 through the bracket 24 regardless of the fixation, the clamping element 36 is equipped with passages 38 that can be located on a concentric ring or cylinder surrounding the clamping element 22.

The catheter 20 is displaceable in the interior space 39 of the bracket 24 along the guiding element 22. Thus, the distance between the distal end of the guiding element 22 and that of the catheter 20 can be changed as needed for the adjustment of its flexibility. As soon as the catheter 20 or its distal end assumes a desired position, the catheter 20 is fixated via the Tuohy burst adaptor 30 or an equivalent element. For this purpose the catheter 20 extends through a rubber element that is shaped like a frustum in its cross section and is squeezed in place with the Tuohy burst adapter 30, as shown in the principal drawing of FIG. 2.

Therefore, the guiding element 22 remains in the catheter 20 even after it has been positioned, always ensuring that the catheter 20 has the desired stiffness during the medical application. The danger according to the state of the art of an undesired shifting of the catheter during the removal of the guiding element also does not occur.

In the embodiments of FIG. 1 to 5, the bracket 24 accommodating the guiding element 22 is designed in form of an elongated pipe element. The bracket 124 has a Y-shaped geometry according to the embodiments in FIG. 6 to 8, consisting of a first leg 128 along the straight line and a second leg 130 at an angle to the first leg and merging with it. Here the filter element 28 extends from the free end of the second leg 136.

The guiding element 22 is fixated in the first leg 128 at its distal cannula end 132. The catheter 20 is displaceable as previously described within the central bore of the first leg 128 relative to the guiding element 22 of the catheter 20 in order to allow a fixation in the desired position.

It can be seen in FIG. 9 that the guiding element 22 in the form of e.g. a pipe consisting of a shape memory alloy such as nickel-titanium alloy is aligned relative to the catheter 20 in such a way that the free ends of guiding element 22 and the catheter 20 end at the desired distance or correspond with each other. The upper drawing in FIG. 9 should indicate a position where the distal ends of the catheter 20 and the guiding element 22 end in the same place. This allocation designated as the first position can be specified during the positioning of the catheter 20.

The guiding element 22 seems to have retreated in the central drawing of FIG. 9, so that the catheter 20 is flexible at the distal end. An even higher flexibility of the distal end of the catheter 20 results from the alignment of the guiding element 22 in relation to catheter 20 in the lower drawing of FIG. 9.

The guiding element 22 runs with its distal end at a distance to the distal end of the catheter 20 according to the distances between the markings A, B, and C in FIG. 9, i.e. within the catheter 20, whereby—as mentioned before—a specific flexibility can be predetermined, whereby the two lower drawings can correspond to a second position of a catheter 20 positioned in an epidural space.

If the guiding element 22 is designated as a pipe in the embodiment in FIG. 9, then the use of a wire, which can also consist of a nickel-titanium alloy, is also possible.

One can see additional options of the solution for the fixation of the guiding element 22 in FIG. 10. A U-shaped or arched end segment 25 can be fixated in the proximal area of the bracket, in particular in an expansion 27, through clamps. Hereby the expansion 27 can be designed as an attachment to or as a pipe element 24, in which the catheter 20 can be displaced.

FIG. 11 provides an alternative embodiment to the drawing in FIG. 10 insofar as the U-shaped bent end segment 25 of the guiding element 22 forms the stop for the catheter 20. The guiding element 22 extends with its free end segment 29 within the pipe segment 24 shown in principal in FIG. 11 so that the free face 31 of the end segment 25 forms the stop for the catheter 20.

Preferred dimensions of catheter 20 and guiding element 22 in form of a pipe or wire are:

Outer diameter of catheter 30: 0.8 mm or 2.3 mm

Diameter of a wire as guiding element 22: between 0.30 and 0.55 mm

Dimensioning of a pipe as guiding outer diameter: 0.4 mm-0.6 mm

element 22 wall thickness: 0.1 mm

The catheter 20 itself preferably consists of a tube element made of polyamide.

Additional preferred designs of the tenet according to the invention can be seen in FIG. 12 to 14 as far as the guiding element 22 in the form of an electricity conducting element, such as a wire, is concerned. The indwelling catheter arrangement according to the invention can also be used for the electrical stimulation of tissue areas, especially in epidural spaces. Here the guiding element 22 is connected with a pole of a power source via a connection 200 in order to acts as a mono-polar electrode. In order to offer simultaneously the option to dispense medication via the catheter 20, in which the guiding element 22 used for the adjustment of the stiffness of the catheter 20 runs, a bracket 202 is provided that has a Y-geometry with an elongated first leg 204 and a second leg 206 running at an angle in the embodiment. The elongated leg 204 has an opening 208 at the end via which through a filter—as previously mentioned—medication can be administered. The end 210 opposite the opening 208 is then connected with the pipe element 212, along which the guiding element 22 runs and that can be displaced as desired relative to the catheter, which is not shown. In this respect, it is referred to the explanations on the embodiments in FIG. 1 to 9, especially to FIG. 9. The medication reaches the distal end of catheter 20 via the clearance between catheter 20 and the guiding element 22 in order to exit from the openings at the distal area when catheter 20 is closed or via its tip if the catheter 20 is not closed.

The guiding element 22 extends through the angled leg 206 of the bracket 202 and is surrounded by the plug element 214 that is inserted fluid-tight at the opening 216 on the end side of the second leg 206. Outside of the bracket 202 or the locking element 214, the guiding element 22 is enclosed by the electrical insulation 218 in order to merge then into the adapter 200 that can be connected with the power source.

In the embodiment of FIG. 13, where the elements corresponding to FIG. 12 are designated with the same reference numbers, the guiding element 22 merges into a connection element 220 such as a metal bushing, which, in turn, is fixated fluid-tight in the second leg 206. From the connecting element 220 the connection runs to the power source.

Pursuant to a special design according to FIG. 14, the guiding element 22 is connected at its proximal end with the electrical conductor 222 whereby the connecting area is injection-molded with plastic. During the injection molding a geometry is formed in the shape of a plug. A corresponding plug 222 is then inserted fluid-tight into the opening 216 of the second leg 206 of the bracket 202, for example with glue.

Through the appropriate measures depicted in FIG. 12 to 14 a safe connection is possible between the guiding element 22 and the electrical connection, whereby the fluid-tight lock takes place outwardly. The guiding element 22 is simultaneously fixated with the plug 214, 222 or the metal bushing 220 in the bracket 202, thereby giving the arrangement the desired steadfastness and the possibility to displace the catheter that is not shown in FIG. 12 to 14 as desired towards the catheter 22 in order to adjust the stiffness of the catheter 20 in the distal area on the one hand and to align the catheter 20 via the distal end of the guiding element 22 on the other hand in such a way that an electrical stimulation can take place in the desired scope if the guiding element 22 acts as an electrode.

In order to achieve the desired scope of the electric current flow between the guiding element 22, meaning its distal end 300 and the tissue to be stimulated, various options are available according to the drawings in FIGS. 15 and 16. The distal area 302 of the catheter 20 can be open so that during the electrical stimulation the distal end 300 of the guiding element 22 extends through the catheter 20 (FIG. 15).

As an alternative, the catheter 20 can be distally closed, as it is possible in the embodiments of FIG. 1 to 10. In order to dispense medication, it is then only necessary to provide one or two openings in the distal area of the catheter 22 closed at the end. In the case of an electrical stimulation in principle more openings are required, as can be seen in FIG. 16. In the embodiment of FIG. 16, the catheter 22 shows several openings in its distal area 304 that are designated for example with the reference numbers 306 and 308. In particular, 8 to 12 openings 306, 308 are provided, in the area of which the guiding element 22 runs in its distal area 300 after the catheter has been positioned.

1. Indwelling catheter arrangement comprising a cannula whose proximal end extends from a receptacle and whose distal end has an opening, in particular, a Tuohy's cut with an opening on the side, as well as a flexible catheter that can be led through the receptacle and through the opening of the cannula, in which in turn a guiding element runs during the positioning of the catheter, characterized in that the guiding element (22) is left in the catheter after the positioning of the catheter (20) and is fixated relative to it in a defined position.

2. Indwelling catheter arrangement according to claim 1 characterized in that the guiding element (22) such as a wire or a pipe remains during the positioning of the catheter (20) relative to it in a first position and is fixated after the positioning of the catheter relative to it in a second position.

3. Indwelling catheter arrangement according to claim 1 characterized in that the indwelling catheter arrangement (10, 100) has a particularly sectionally pipe-like bracket (24, 124), in which on one hand the guiding element (22) is fixated and on the other hand the catheter (20) runs with its proximal end, which can be displaced in the bracket

4. Indwelling catheter arrangement claim 1 characterized in that the catheter 20) is fixated via a claming element (40).

5. Indwelling catheter arrangement claim 1 characterized in that the catheter (20) is fixated via a Tuohy burst adapter (30) or an equivalent element.

6. Indwelling catheter arrangement claim 1 characterized in that the bracket (24, 124) has a fluid connection (26, 126), upstream of which a filter (28) can be installed.

7. Indwelling catheter arrangement claim 1 characterized in that the bracket (124) has a Y- or V-shaped geometry with a first leg (128) running along a straight line and a second leg (130) running at an angle to the first leg and merging with it and that the guiding element (22) is fixated in the first leg and that in it the proximal end of the catheter (20) surrounding the guiding element is displaceable arranged.

8. Indwelling catheter arrangement claim 1 characterized in that the guiding element (22) is held in the bracket (24) by a clamping element (36), which, in turn, has at least one fluid passage (38).

9. Indwelling catheter arrangement claim 1 characterized in that the fluid passage (38) is formed by concentric passages (28) around the guiding element (22).

10. Indwelling catheter arrangement claim 1 characterized in that the catheter (20) is fastened by a squeeze fixation.

11. Indwelling catheter arrangement claim 1 characterized in that the guiding element (22) is fixated in the clamping element (24, 124), in particular by glue.

12. Indwelling catheter arrangement claim 1 characterized in that the guiding element (22) is fixated in a wall (132) of the bracket (124) remote to the cannula (12).

13. Indwelling catheter arrangement claim 1 characterized in that the guiding element is fixated in the proximal area of the bracket through clamps, in particular through clamps of a bent end segment of the guiding element.

14. Indwelling catheter arrangement claim 1 characterized in that the guiding element (22) consists of or contains a shape memory alloy such as a nickel-titanium alloy.

15. Indwelling catheter arrangement claim 1 characterized in that the guiding element (22) has the form of a wire with a diameter ranging from 0.30 mm to 0.55 mm.

16. Indwelling catheter arrangement claim 1 characterized in that the catheter (20) has an outer diameter between 0.8 mm and 2.3 mm.

17. Indwelling catheter arrangement claim 1 characterized in that the guiding element (22) is a pipe element whose outer diameter is between approximately 0.4 mm and 0.6 mm and whose wall thickness is approximately 0.1 mm.

18. Indwelling catheter arrangement claim 1 characterized in that the catheter (20) consists preferably of polyamide, poly-methane or silicon.

19. Indwelling catheter arrangement claim 1 characterized in that the cannula is a split cannula.

20. Indwelling catheter arrangement claim 1 characterized in that the guiding element (22) has a U-shape for the fixation at the end side, whereby the free face surface (31) of the bent guiding element segment (29) is the proximal stop for the displaceable catheter (20) relative to the guiding element.

21. Indwelling catheter arrangement claim 1 characterized in that the guiding element (22) is an electrode that can be connected to a power source, which can be connected in the distal area (302, 304) of the catheter (20) via at least one opening (303, 306, 308) with tissue or with tissue fluids.

22. Indwelling catheter arrangement claim 1 characterized in that the catheter (22) is distally closed and is equipped at a distance at the distal end with at least one, preferably with several openings (306, 308).

23. Indwelling catheter arrangement claim 1 characterized in that the guiding element (22) extends through the catheter (20) at its distal end (302).

24. Indwelling catheter arrangement claim 1 characterized in that the distal area (300) of the guiding element (22) is always within the catheter (22).

25. Indwelling catheter arrangement claim 1 characterized in that the catheter (20) has several openings in the distal area (304), preferably eight to twelve openings (306, 308).

26. Indwelling catheter arrangement claim 1 characterized in that the guiding element (22) is fixated in a bracket (202) with a preferably V- or Y-geometry, that the bracket has a straight line first leg (104) along which the guiding element (22) and the catheter (20), surrounding said element and being displaceable thereto, run in sections or in its direction, and that the bracket comprises a second leg (206) that is at an angle to the first leg where the guiding element is fixated.

27. Indwelling catheter arrangement claim 1 characterized in that the guiding element (22) is steadfastly fixated in a bracket (202) or in its second leg (206) via a plug (214, 222) that is inserted fluid-proof in an opening (216)

28. Indwelling catheter arrangement according to at least one of the previous claims characterized in that the plug (214, 222) is fixated fluid-tight, such as glued, into the opening (216) of the second leg (206).

29. Indwelling catheter arrangement claim 1 characterized in that the connection between the guiding element (22) and the electrical connection (222) such as an electrical conductor is injection molded into a plug shape.

30. Indwelling catheter arrangement claim 1 characterized in that the guiding element (22) extends from a metal bushing (220) that is inserted into the second leg (206) or its opening (216) in a fluid-tight manner.

31. Indwelling catheter arrangement claim 1 characterized in that medication can be dispensed via the first leg (204) of the bracket (202).
